(19) United States
(12) Patent Application Publication
(10) Pub. No.: US 2022/0184247 A1
(43) Pub. Date: Jun. 16, 2022

(54) MICROPHONE DISINFECTOR

(71) Applicant: AUDIO-TECHNICA CORPORATION, Tokyo (JP)

(72) Inventors: Ren UESUGI, Fukui (JP); Shota FUJII, Fukui (JP); Noriyuki MATSUDA, Fukui (JP)

(21) Appl. No.: 17/546,230

(22) Filed: Dec. 9, 2021

(30) Foreign Application Priority Data
Dec. 10, 2020 (JP) 2020-205399

(51) Int. Cl. A61L 2/08; A61L 2/24

(52) U.S. Cl. CPC A61L 2/085; A61L 2/24; A61L 2202/121; A61L 2202/14; A61L 2202/11

(57) ABSTRACT

A microphone disinfector according to the present invention includes a disinfection part that disinfects and sterilizes a microphone M, light emitting parts that emit infrared light to a region where the microphone is arranged, light receiving parts that receive reflected light of the infrared light and generate an electric signal based on the received light, a determination part that determines the presence or absence of the microphone in the region based on the electric signal, and a disinfection control part that controls an operation of the disinfection part based on a determination result of the determination part. The infrared light is pulsed light with a predetermined blinking light emission period. The determination part determines whether an amplitude value for each period of the electric signal is equal to or greater than a first threshold value, and determines the presence or absence of the microphone based on whether a first state, in which the amplitude value is equal to or greater than the first threshold value, is continuous for a first time.

### TECHNICAL FIELD

The present invention relates to a microphone disinfector.

### BACKGROUND ART

A handheld microphone (hereinafter, referred to as a “microphone”) is used by an unspecified number of people in, for example, karaoke shops, classrooms of universities and cram schools, conference rooms, and the like. Such a microphone includes a mesh-shaped sound collection part (head part) and a grip part extending cylindrically from the sound collection part. The microphone is used in a state in which the grip part is gripped by the hand of a user and the sound collection part is close to the mouse of the user. Therefore, the grip part is easily contaminated by the sweat and dirt of the user, and the sound collection part is easily contaminated by saliva scattered from the mouse of the user. Consequently, the microphone is disinfected and cleaned according to the frequency of use or periodically. As described above, since the grip part of the microphone has a cylindrical shape, the grip part can be disinfected and cleaned relatively easily. However, since the head part has a mesh shape, a surface of the head part can be disinfected and cleaned relatively easily, but an inner side or an inner part of the mesh cannot be easily disinfected and cleaned.

Technologies of automatically disinfecting and sterilizing the head part of a microphone by using ultraviolet rays have been disclosed (for example, see Japanese Utility Model Registration No. 3227849, Japanese Unexamined Utility Model Publication No. 7-29599, Japanese Unexamined Patent Application Publication No. 2011-97511, and Japanese Unexamined Patent Application Publication No. 8-265890).

In the technologies disclosed in Japanese Utility Model Registration No. 3227849, Japanese Unexamined Utility Model Publication No. 7-29599, Japanese Unexamined Patent Application Publication No. 2011-97511, and Japanese Unexamined Patent Application Publication No. 8-265890, the head part of the microphone held by a disinfection apparatus or instrument is disinfected and sterilized by irradiating the head part with ultraviolet rays. In these technologies, the on/off of ultraviolet irradiation is controlled by the on/off of a switch (Japanese Utility Model Registration No. 3227849, Japanese Unexamined Utility Model Publication No. 7-29599, and Japanese Unexamined Patent Application Publication No. 2011-97511) or detecting the presence or absence of the microphone (Japanese Utility Model Registration No. 3227849 and Japanese Unexamined Patent Application Publication No. 8-265890).

In general, a method of detecting the presence or absence of an article in a predetermined region is classified into a contact type method via physical contact as disclosed in Japanese Unexamined Patent Application Publication No. 8-265890 and a non-contact type method using ultrasonic waves or electromagnetic waves without physical contact. Of these, a non-contact type infrared sensor using infrared light is widely used because it is relatively inexpensive and less likely to break down than the contact type.

However, the microphone is used in various external environments such as a room like a karaoke room where the bright-dark and blinking of lighting are extremely switched, a room like a classroom where sunlight comes in, and a room like a conference room where it is easy to maintain constant brightness. Furthermore, in many rooms where the microphone is used, electric appliances such as televisions and air conditioners operated by infrared remote controllers are installed. In such environments, an infrared sensor may be affected by external light or infrared light from the infrared remote controller. Therefore, a malfunction or unstable operation of the infrared sensor may occur.

When the head part of the microphone is disinfected and sterilized, the head part of the microphone is exposed to ultraviolet rays or ozone. Furthermore, the head part has a mesh shape. Therefore, the head part is not suitable as a part to be irradiated with the infrared light of the infrared sensor, and the grip part of the microphone is irradiated with the infrared light. However, since the grip part has a cylindrical shape, the infrared light is likely to be diffusely reflected off the surface of the grip part and the intensity of reflected light received by a light receiving part of the infrared sensor is reduced. Furthermore, the surface state (for example, color, material, surface treatment, presence or absence of dirt and scratch, a combination thereof, and the like) of the grip part of the microphone is various, and the intensity of the reflected light received by the light receiving part of the infrared sensor may vary depending on the surface state of the grip part. In order to solve these problems, various methods (for example, a method of increasing the output of the infrared light, a method of shortening a distance between the microphone and a light emitting part of the infrared light, a method of blocking external light by a case and the like, and so on) are required. However, each of these methods may have various corresponding restrictions (for example, restrictions on power consumption, arrangement and positional relationship between the microphone and the infrared sensor, case design and arrangement, and the like). As described above, many problems exist in stably detecting the presence or absence of the microphone by the infrared light (electromagnetic waves).

### SUMMARY OF INVENTION

### Technical Problem

An object of the present invention is to solve the problem described above and to provide a microphone disinfector capable of stably detecting the presence or absence of a microphone by infrared light.

### Solution to Problem

A microphone disinfector according to the present invention includes a disinfection part that disinfects and sterilizes a microphone, a light emitting part that emits infrared light to a region where the microphone is arranged, a light receiving part that receives reflected light of the infrared light and generates an electric signal based on the received light, a determination part that determines presence or absence of the microphone in the region based on the electric signal, and a disinfection control part that controls an operation of the disinfection part based on a determination result of the determination part. The infrared light is pulsed light with a predetermined blinking light emission period, and the determination part determines whether an amplitude value for each period of the electric signal is equal to or greater than a first threshold value, and determines the presence or absence of the microphone based on whether a first state, in which the amplitude value is equal to or greater than the first threshold value, is continuous for a first time.

### Advantageous Effects of the Invention

The present invention can stably detect the presence or absence of a microphone by infrared light.

### BRIEF DESCRIPTION OF DRAWINGS

FIG. 1 is a perspective view illustrating an embodiment of a microphone disinfector according to the present invention.

FIG. 2 is an exploded perspective view of the microphone disinfector in FIG. 1.

FIG. 3 is a functional block diagram of the microphone disinfector in FIG. 1.

FIG. 4 is a front view of the microphone disinfector in FIG. 1. FIG. 5 is a cross-sectional view of the microphone disinfector taken along line AA in FIG. 4.

FIG. 6 is an enlarged cross-sectional view of the vicinity of a first sensor provided in the microphone disinfector taken along line BB in FIG. 4.

FIG. 7 is an enlarged front view of the vicinity of the first sensor in FIG. 6.

FIG. 8 is a graph illustrating the radiation intensity of a light receiving surface of a light receiving element provided in the microphone disinfector in FIG. 1 when the configuration of a long groove provided in the first sensor in FIG. 7 is changed.

FIG. 9 is an enlarged left side view of the microphone disinfector in FIG. 1.

FIG. 10 is a flowchart illustrating an example of an operation of the microphone disinfector in FIG. 1.

FIG. 11 is a flowchart illustrating a first determination process included in the operation in FIG. 10.

FIG. 12 is a schematic view illustrating an example of an electric signal from the light receiving element provided in the microphone disinfector in FIG. 1, in the first determination process in FIG. 10.

FIG. 13 is an enlarged schematic view illustrating an example of the electric signal from the light receiving element in FIG. 12.

FIG. 14 is a flowchart illustrating a second determination process included in the operation in FIG. 10.

FIG. 15 is a schematic view illustrating an example of an electric signal from the light receiving element in the second determination process in FIG. 14.

FIG. 16 is a flowchart illustrating an output adjustment process included in the operation in FIG. 10.

FIG. 17 is a schematic view illustrating an example of an electric signal from the light receiving element in the output adjustment process in FIG. 16.

FIG. 18 is a flowchart illustrating a disinfection and sterilization process included in the operation in FIG. 10.

### DESCRIPTION OF EMBODIMENTS

Hereinafter, embodiments of a microphone disinfector (hereinafter, referred to as “present disinfector”) according to the present invention will now be described with reference to the attached drawings. In each drawing, the same members and components are designated by the same reference numerals, and redundant description thereof will be omitted.

### Microphone Disinfector

### Configuration of Microphone Disinfector

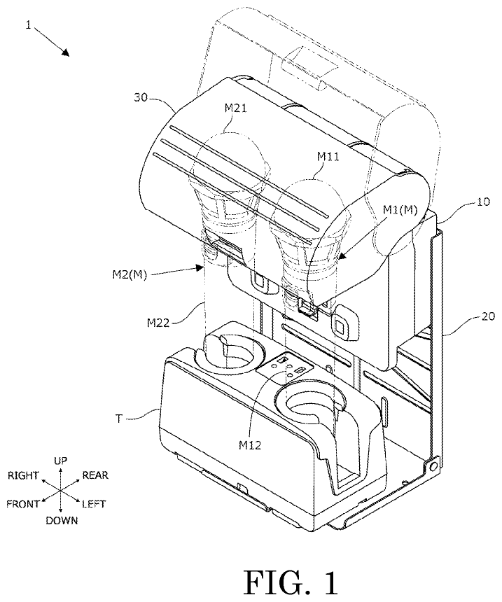

FIG. 1 is a perspective view illustrating an embodiment of the present disinfector.

FIG. 1 also illustrates microphones M1 and M2 charged by a charger T for convenience of description.

The present disinfector 1 disinfects and sterilizes head parts M11 and M21 of the microphones M1 and M2. The present disinfector 1 includes a body 10, a bracket 20, and a shade 30.

The microphones M1 and M2 are handheld microphones in which grip parts M12 and M22 are used by being gripped by the hand of a user in, for example, a karaoke shop and the like. The microphones M1 and M2 are, for example, charged while standing upright on a charger T and are disinfected and sterilized by the present disinfector 1. In the following description, when the microphones M1 and M2 are not distinguished, the microphones M1 and M2 are referred to as a microphone M.

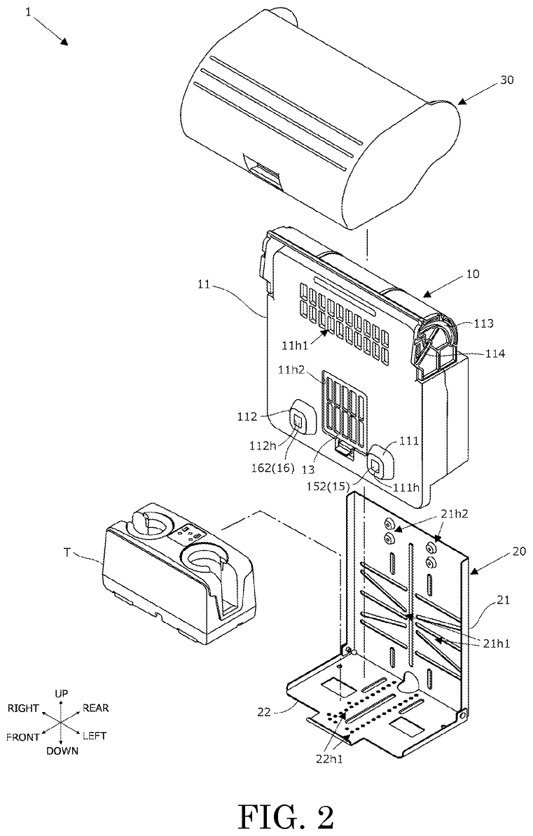

FIG. 2 is an exploded perspective view of the present disinfector 1.

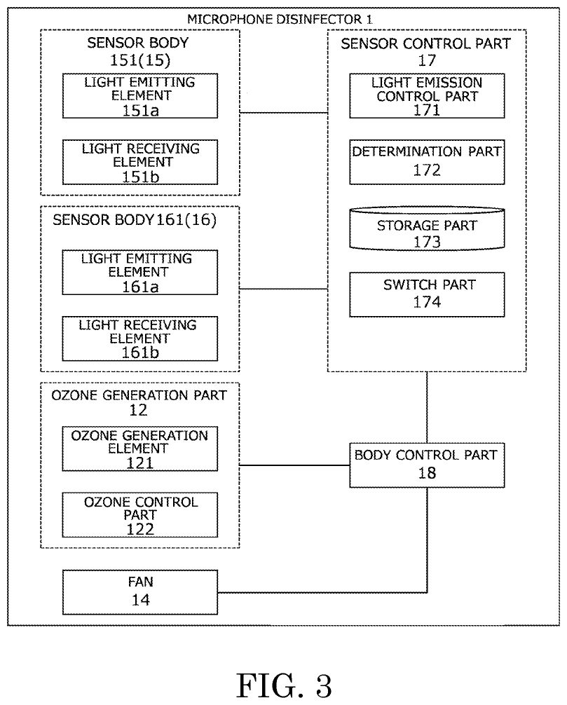

FIG. 3 is a functional block diagram of the present disinfector 1.

FIG. 2 also illustrates the charger T for convenience of description.

The body 10 blows air including ozone (hereinafter, referred to as “ozone wind”) for disinfecting and sterilizing the microphone M. The body 10 includes a housing 11, an ozone generation part 12, a filter 13, a fan 14, a first sensor 15, a second sensor 16, a sensor control part 17, and a body control part 18.

The housing 11 accommodates the ozone generation part 12, the fan 14, the first sensor 15, the second sensor 16, the sensor control part 17, and the body control part 18. The housing 11 has a substantially hollow rectangular parallelepiped shape. In the side view, a rear upper end of the housing 11 has a semicircular shape.

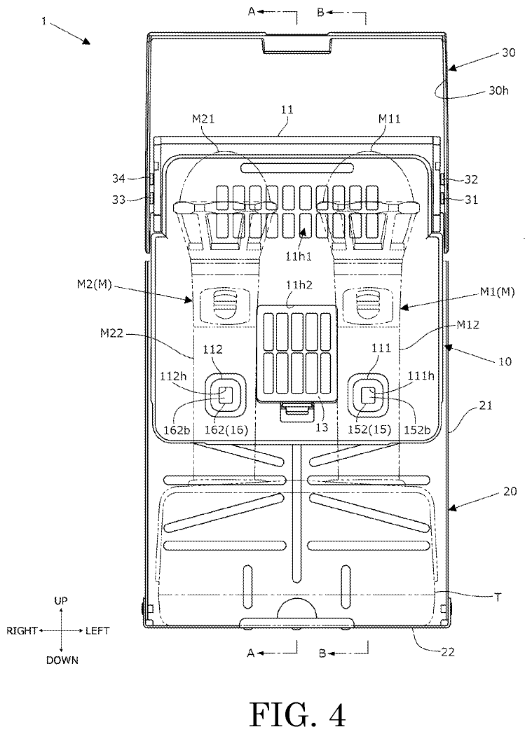

FIG. 4 is a front view of the present disinfector 1.

FIG. 5 is a cross-sectional view of the present disinfector 1 taken along line AA in FIG. 4.

In FIG. 4, the microphones M1 and M2 and the charger T are indicated by a two dot chain line for convenience of description. In FIG. 5, the flow of air is indicated by white arrows, the flow of ozone is indicated by small black arrows, and the flow of ozone wind is indicated by black arrows.

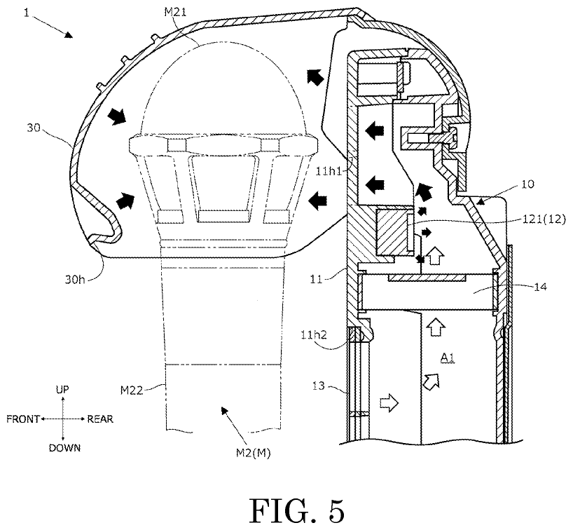

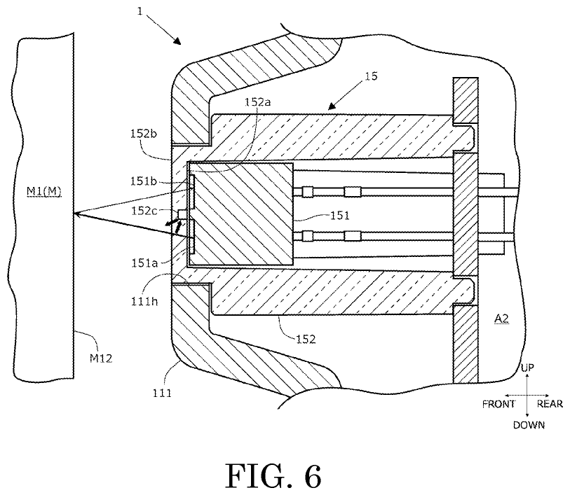

An internal space of the housing 11 is roughly divided into a ventilation chamber Al arranged (disposed) in the center of the housing 11 in the left-right direction and a board chamber A2 (see FIG. 6 and the same applies below) arranged to surround the left, right, and lower sides of the ventilation chamber A1. The housing 11 includes a plurality of blowing ports 11h1, an intake port 11h2, two protrusions 111 and 112, two openings 111h and 112h, and four rails 113 and 114 (see FIG. 2, two rails on the right side are not illustrated, and the same applies below).

The blowing ports 11h1 are openings for blowing the ozone wind into a space inside the shade 30. The respective blowing ports 11h1 are arranged side by side in two rows at the upper part of a front surface of the housing 11.

The intake port 11h2 is an opening for intaking air serving as the ozone wind into the ventilation chamber A1. The intake port 11h2 is arranged at the lower part of the front surface of the housing 11.

The protrusion 111 is a protrusion in which the first sensor 15 is arranged, and the protrusion 112 is a protrusion in which the second sensor 16 is arranged. The protrusions 111 and 112 are arranged at the lower left and right parts of the front surface of the housing 11, and face the microphones M1 and M2 standing upright on the charger T.

The opening lllh is an opening for allowing the passage of infrared light from the first sensor 15 and light including reflected light of the infrared light from an exterior (installation environment of the present disinfector 1). The opening 112h is an opening for allowing the passage of infrared light from the second sensor 16 and light including reflected light of the infrared light from an exterior. The opening 111h is arranged on a top surface (front surface) of the protrusion 111, and the opening 112h is arranged on a top surface (front surface) of the protrusion 112.

The rails 113 and 114 are grooves for guiding the opening and closing of the shade 30. The rail 113 is an arc-shaped long groove along the rear upper end of the housing 11. The rail 114 is a long groove extending in a direction intersecting the rail 113. The rails 113 and 114 are arranged at the upper part of a left side surface of the housing 11. The rail 114 is arranged radially inside the rail 113. The other two rails (not illustrated) are arranged at the upper part of a right side surface of the housing 11 in the same manner as the rails 113 and 114.

Referring now back to FIG. 3 and FIG. 5, the ozone generation part 12 generates ozone for disinfection and sterilization and disinfects and sterilizes the microphone M by the ozone wind in a non-contact manner. The ozone generation part 12 is a known ozone generation module (ozonizer) including an ozone generation element 121 and an ozone control part 122. The ozone generation part 12 is an example of a disinfection part in the present invention. The ozone generation element 121 is accommodated in the ventilation chamber A1 and is arranged above the fan 14 to be described below. The ozone control part 122 is accommodated in the board chamber A2.

The filter 13 filters the air serving as the ozone wind. The filter 13 is attached to the intake port 11h2.

The fan 14 intakes and blows the air serving as the ozone wind. The fan 14 is accommodated in the ventilation chamber A1 and is arranged near the center of the ventilation chamber A1.

FIG. 6 is an enlarged cross-sectional view of the vicinity of the first sensor of the present disinfector 1 taken along line BB in FIG. 4.

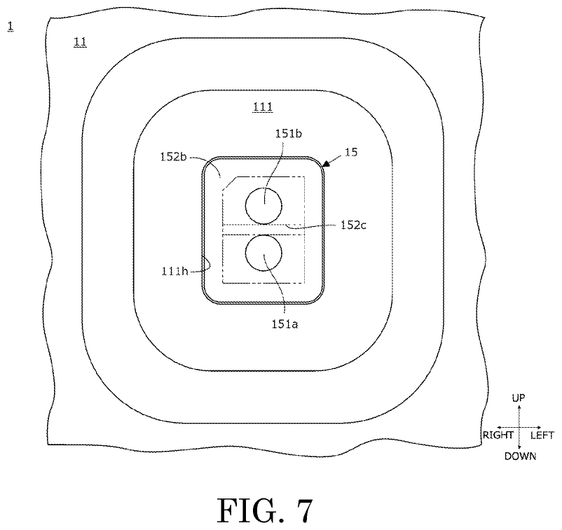

FIG. 7 is an enlarged front view of the vicinity of the first sensor 15 of the present disinfector 1.

In FIG. 6, a part of the behavior of infrared light from a light emitting element 151a to be described below is indicated by arrows for convenience of description.

The first sensor 15 detects the presence or absence of the microphone M (microphone M1 in the present embodiment). The first sensor 15 includes a sensor body 151 and a sensor cover 152. The first sensor 15 is arranged in the protrusion 111.

The sensor body 151 is, for example, a known infrared sensor in which the light emitting element 151a and a light receiving element 151b are integrated (integrally configured) as one chip. That is, the sensor body 151 includes the light emitting element 151a and the light receiving element 151b as a single body. The light emitting element 151a and the light receiving element 151b are vertically arranged side by side on a front surface of the sensor body 151.

The light emitting element 151a blinks and emits infrared light, which is pulsed light with a constant light emission period, to a determination region to be described below. The blinking frequency of the infrared light is set to a frequency extremely lower (for example, lower by about two digits) than the carrier frequency (for example, an infrared remote controller: 38 kHz and sound transmission: 2 MHz to 6 MHz) of infrared light (hereinafter, referred to as “transmission infrared light”) used as a carrier wave for general wireless transmission. In the present embodiment, the blinking frequency of the infrared light is 25 Hz and the light emission period of the infrared light is 0.04 seconds. The light emitting element 151a is an example of a light emitting part in the present invention.

Note that the blinking frequency of the infrared light from the light emitting element is not limited to 25 Hz as long as it is extremely lower (for example, about two digits) than the carrier frequency band of the transmission infrared light. That is, for example, the blinking frequency band of the infrared light is preferably about 1 kHz or less (for example, 5 Hz to 1 kHz), is more preferably about 500 Hz or less (for example, 5 Hz to 500 Hz), and is more preferably about 10 Hz to 100 Hz based on the first number of consecutive times (the number of second consecutive times) to be described below.

The light receiving element 151b receives the reflected light of the infrared light from the light emitting element 151a, and generates an electric signal corresponding to the received light. The electric signal corresponding to the reflected light is a pulsed electric signal having the same period as that of the infrared light. The light receiving element 151b is an example of a light receiving part in the present invention.

Here, in general, the use of a cover member that covers a light emitting element and a light receiving element in an infrared sensor causes a decrease in the sensitivity of the infrared sensor. Particularly, in a one-chip type infrared sensor such as the sensor body 151 according to the present embodiment, a phenomenon occurs in which infrared rays from the light emitting element 151a are reflected by the surface (inner surface or outer surface) of the cover member and received by (incident on) the light receiving element 151b. Therefore, in general, no cover member is used in the infrared sensor. However, since the present disinfector 1 may also be installed in a store accompanying food and drink such as a karaoke store, the food and drink may adhere to the infrared sensor. Furthermore, when the microphone M is set in the present disinfector 1, a person's hand may contact the infrared sensor. The contact of the human body or food and drink to the infrared sensor causes a decrease in the sensitivity of the infrared sensor or a failure thereof. Therefore, the present disinfector 1 includes the sensor cover 152 that protects the sensor body 151.

The sensor cover 152 protects the sensor body 151 from the contact of the human body, adhesion of foreign matters (dust and food and drink), and the like. The sensor cover 152 is made of, for example, a transparent synthetic resin such as polycarbonate. The sensor cover 152 covers at least the front side of each of the light emitting element 151a and the light receiving element 151b. The sensor cover 152 includes a first surface 152a, a second surface 152b, and a long groove 152c.

The first surface 152a is an inner surface facing the front surface of each of the light emitting element 151a and the light receiving element 151b, and the second surface 152b is an outer surface parallel to the first surface 152a. Since each of the first surface 152a and the second surface 152b is finished to be a mirror surface, for example, in order to restrain the reflection of the infrared light. The sensor cover 152 is fitted into the opening 111h so that the second surface 152b is continuous with the top surface of the protrusion 111.

The long groove 152c prevents the infrared light reflected in the sensor cover 152 from being incident on the light receiving element 151b side, as indicated by the black arrows in FIG. 6. The long groove 152c is arranged in the first surface 152a along the left-right direction so as to separate the light emitting element 151a and the light receiving element 151b in the front view. The long groove 152c is recessed in a rectangular shape from the first surface 152a toward the second surface 152b in the cross-sectional view parallel to a short direction (vertical direction) of the long groove 152c. In the present embodiment, the depth of the long groove 152c is 3/5 (that is, 0.6 mm) of the thickness of the sensor cover 152 (thickness 1 mm between the first surface 152a and the second surface 152b).

Note that the depth of the long groove in the present invention is not limited to the present embodiment as long as it has an effect of preventing the infrared light reflected in the sensor cover from being incident on the light receiving part. Here, when the depth of the long groove is 2/5 or more of the thickness of the sensor cover, the effect is exhibited, and the effect is improved as the depth increases whereas the strength of the sensor cover is reduced. In the present invention, it is sufficient if the thickness of the sensor cover is, for example, 0.5 mm to 1.5 mm and preferably 0.8 mm to 1.2 mm based on the balance between the strength of the sensor cover and the reflection in the sensor cover.

Furthermore, the cross-sectional shape of the long groove in the present invention is not limited to the rectangular shape. That is, for example, the long groove may have a semicircular, U-shaped, or V-shaped cross-sectional shape.

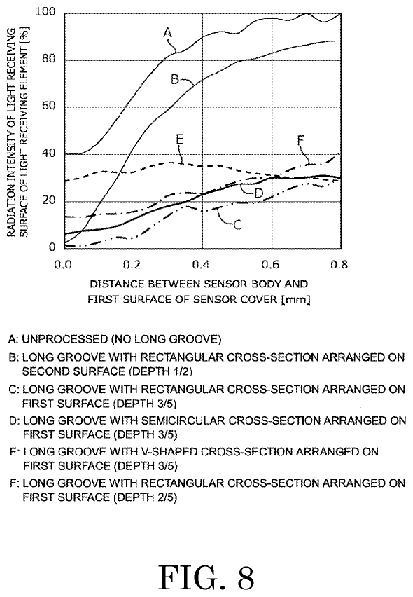

FIG. 8 is a graph illustrating a relationship between the distance between the sensor body 151 and the first surface 152a of the sensor cover 152 and the radiation intensity of a light receiving surface of the light receiving element 151b when the configuration of the long groove 152c is changed.

FIG. 8 illustrates an optical simulation result when the radiation intensity of the light receiving surface having a distance of 0.8 mm is 100%. In FIG. 8, the thickness of the sensor cover 152 is 1 mm. FIG. 8 illustrates that no effect is exhibited even though the long groove is arranged in the second surface 152b (“B” in FIG. 8). Furthermore, FIG. 8 illustrates that the effect is improved as the depth of the long groove increases (“C” and “F” in FIG. 8). Moreover, FIG. 8 illustrates that the effect is improved in the order of V-shaped, semicircular, and rectangular cross-sectional shapes (“E”, “D”, and “C” in FIG. 8).

In this way, in the present disinfector 1, although the first sensor 15 includes the sensor cover 152, a decrease in the sensitivity of the sensor body 151 is restrained as much as possible. Furthermore, since the human body and foreign matters come into contact with only the second surface 152b of the sensor cover 152, the sensitivity of the sensor body 151 is maintained only by cleaning the second surface 152b. That is, in the present disinfector 1, a decrease in the sensitivity and failure of the sensor body 151 are restrained, resulting in the improvement of maintainability

Referring now back to FIG. 3 and FIG. 4, the second sensor 16 detects the presence or absence of the microphone (microphone M2 in the present embodiment). The second sensor 16 includes a sensor body 161 and a sensor cover 162. The second sensor 16 is arranged in the protrusion 112.

The second sensor 16 has the same configuration as that of the first sensor 15. That is, the sensor body 161 includes a light emitting element 161a and a light receiving element 16 lb. The light emitting element 161a is an example of a light emitting part in the present invention, and the light receiving element 161b is an example of a light receiving part in the present invention. The sensor cover 162 includes a first surface (not illustrated), a second surface 162b, and a long groove (not illustrated). The sensor cover 162 is fitted into the opening 112h so that the second surface 162b is continuous with the top surface of the protrusion 112.

The sensor control part 17 controls the operations of the light emitting elements 151a and 161a, and processes the electric signals from the light receiving elements 151b and 161b. The sensor control part 17 is, for example, a microcontroller having a central processing unit (CPU), a read only memory (ROM), a random access memory (RAM), and the like. The sensor control part 17 includes a light emission control part 171, a determination part 172, a storage part 173, and a switch part 174.

The light emission control part 171 controls the light emission output of the infrared light emitted from the light emitting elements 151a and 161a based on a determination result of the determination part 172. The detailed operation of the light emission control part 171 will be described below.

The determination part 172 determines the presence or absence of the microphones M1 and M2 in a region (hereinafter, referred to as “determination region”), where the microphones M1 and M2 are arranged, based on the electric signals from the light receiving elements 151b and 161b. The detailed operation of the determination part 172 will be described below.

The storage part 173 stores information necessary for the operation of the sensor control part 17. Details of the information stored in the storage part 173 will be described below.

The switch part 174 switches a determination execution time. The detailed operation of the switch part 174 will be described below.

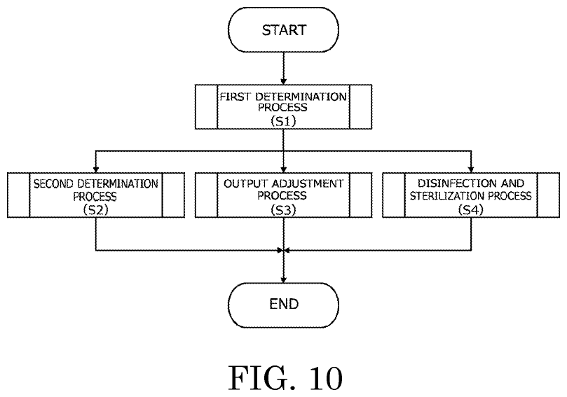

The “determination execution time” is a time during which the determination part 172 continuously executes a first determination process S1 (see FIG. 10) to be described below. The determination execution time includes a first determination execution time at which the determination part 172 always executes the first determination process S1 and a second determination execution time at which the determination part 172 executes the first determination process S1 periodically (for example, at intervals of 0.5 seconds). The determination execution time is set and selected by, for example, a user of the present disinfector 1.

The body control part 18 controls the overall operations of the body 10 (for example, the operations of the ozone generation part 12 and the fan 14). The body control part 18 is, for example, a microcontroller common to the sensor control part 17. The body control part 18 is an example of a disinfection control part in the present invention.

Note that each of the sensor control part and the body control part in the present invention may not be configured by a common microcontroller. That is, for example, each of the sensor control part and the body control part in the present invention may be configured by an individual microcontroller or processor, or may be configured by an individual circuit that executes a predetermined process.

Referring now back to FIG. 1, FIG. 2, and FIG. 5, the bracket 20 supports the body 10. The bracket 20 is made of, for example, metal such as stainless steel. The bracket 20 includes a back panel 21 and a bottom panel 22. The back panel 21 has a substantially rectangular plate shape that is long in the vertical direction. The bottom panel 22 has a substantially rectangular plate shape that extends forward from a lower end of the back panel 21 and is long in the left-right direction. That is, the bracket 20 has an L shape in the side view. The back panel 21 includes a plurality of mounting slits 21h1 through which screws (not illustrated) for mounting the present disinfector 1 on a wall, a rack, and the like are inserted, and a plurality of mounting holes 21h2 through which screws (not illustrated) for mounting the body 10 on the bracket 20 are inserted. The bottom panel 22 includes a plurality of mounting holes 22h1 through which screws (not illustrated) for mounting the present disinfector 1 are inserted.

The body 10 is mounted on the front surface of the back panel 21 of the bracket 20, for example. At this time, the charger T is mounted on the upper surface of the bottom panel 22 of the bracket 20. As a consequence, when the microphones M1 and M2 are charged, the grip part M12 of the microphone M1 is arranged in front of the first sensor 15 and the grip part M22 of the microphone M2 is arranged in front of the second sensor 16. That is, the determination region is a region through which the infrared light from the first sensor 15 (second sensor 16) passes, and is a region where the grip part M12 (M22) of the microphone M1 (M2) being charged is arranged.

Note that the body may be mounted on the bottom panel of the bracket.

The shade 30 allows the ozone wind from the blowing ports 11h1 to stay around the head parts M11 and M21 of the microphones M1 and M2 that are disinfected and sterilized. The shade 30 is made of, for example, a transparent synthetic resin such as polycarbonate. The shade 30 is hollow and has a substantially oval columnar shape in the side view. The shade 30 has an opening 30h at the bottom thereof, into which the body 10 and the microphones M1 and M2 can be inserted.

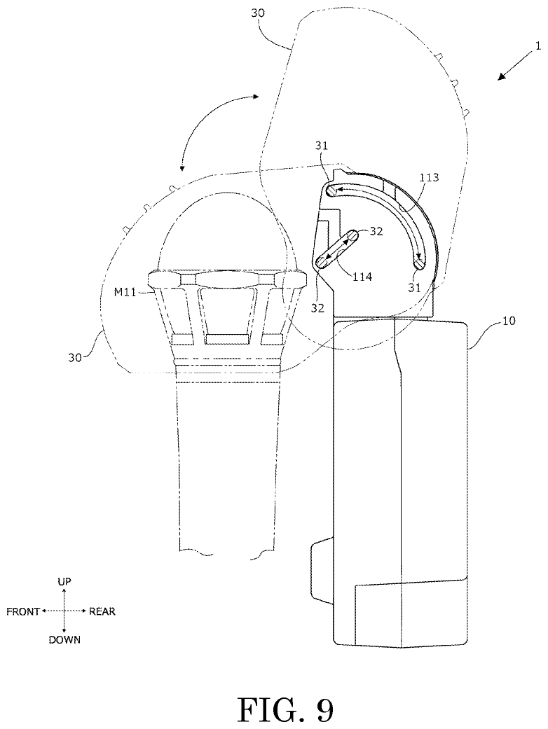

FIG. 9 is an enlarged left side view of the microphone disinfector 1.

In FIG. 9, the shade 30 in an opened and closed state is indicated by a two dot chain line for convenience of description.

Two protrusions 31 and 32 are arranged on the inner surface of a left side surface of the shade 30. Two protrusions 33 and 34 are arranged on the inner surface of a right side surface of the shade 30 at positions facing the protrusions 31 and 32 (see FIG. 4 and the same applies below). The protrusions 31 and 32 are columnar protrusions corresponding to the rails 113 and 114 on the left side surface of the housing 11, and the protrusions 33 and 34 are columnar protrusions corresponding to the rails (not illustrated) on the right side surface of the housing 11. The shade 30 is mounted on the body 10 by being fitted to the rails 113 and 114 (rails on the right side surface are not illustrated and the same applies below) corresponding to the left protrusions 31 and 32 and the right protrusions 33 and 34.

### Opening and Closing of Shade

The opening and closing of the shade 30 will now be described taking the protrusions 31 and 32 and the rails 113 and 114 as an example. The protrusion 31 is slidable in the rail 113 having an arc-shape and the protrusion 32 is slidable in the rail 114 having a straight line shape. The shade 30 is opened and closed when the protrusions 31 and 32 slide in the rails 113 and 114. Specifically, the shade 30 is closed when the protrusions 31 and 32 are located at the front ends of the rails 113 and 114, and is opened when the protrusions 31 and 32 are located at the rear ends of the rails 113 and 114. At this time, the protrusion 32 slides backward-obliquely-upward along the rail 114 and the protrusion 31 slides backward downward along the rail 113. Therefore, the shade 30 is opened and closed while its rotation axis moves. As a consequence, when the shade 30 is closed, the shade 30 protrudes to the front of the body 10 so as to cover the front of the blowing ports 11h1 (see FIG. 2) of the body 10. On the other hand, when the shade 30 is opened, the shade 30 protrudes above the body 10 without protruding rearward from the body 10. According to such a configuration, for example, when the present disinfector 1 is mounted on a wall, the shade 30 is opened and closed without interference from the wall.

### Operation of Microphone Disinfector

Next, the operation of the present disinfector 1 will now be described with reference to FIG. 1 to FIG. 3 by taking the determination by the first sensor 15 regarding the presence or absence of the microphone M1 as an example.

FIG. 10 is a flowchart illustrating an example of the operation of the present disinfector 1.

The present disinfector 1 executes, for example, the first determination process S 1, a second determination process S2, an output adjustment process S3, and a disinfection and sterilization process S4. The second determination process S2, the output adjustment process S3, and the disinfection and sterilization process S4 are executed after the first determination process S1.

### First Determination Process

The “first determination process S1” is a process of determining the presence or absence of the microphone M in the determination region when the microphone M is not in the determination region. That is, the first determination process S1 is a process of determining whether the microphone M to be disinfected has been set in the present disinfector 1. In the following description, it is assumed that the present disinfector 1 determines the presence or absence of the microphone M1 in the determination region by emitting infrared light from the light emitting element 151a toward the determination region and monitoring an electric signal from the light receiving element 151b.

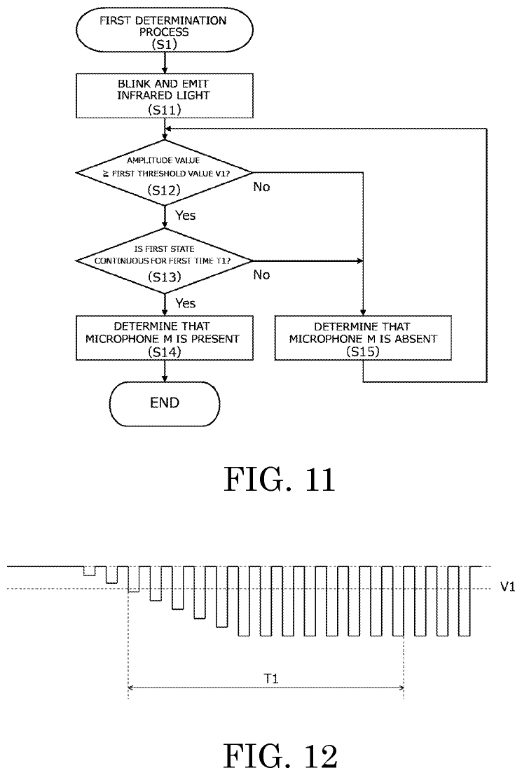

FIG. 11 is a flowchart illustrating the first determination process S1. FIG. 12 is a schematic view illustrating an example of an electric signal from the light receiving element 151b in the first determination process S1.

First, the light emission control part 171 allows the light emitting element 151a to blink and emit infrared light that is pulsed light with a constant light emission period (S11). As described above, in the present embodiment, since the blinking frequency of the infrared light is 25 Hz, the blinking light emission period is 0.04 seconds. Since the grip part M12 of the microphone M1 has a cylindrical shape, the infrared light is diffusely reflected off the surface of the grip part M12. Therefore, the intensity of reflected light of the infrared light reflected by the grip part M12 is lower than that of the infrared light from the light emitting element 151a. Consequently, in the first determination process S1, the light emission output of the infrared light is set to a magnitude that allows the light receiving element 151b to receive reflected light with a sufficient intensity (intensity at which an amplitude value to be described below is equal to or greater than a first threshold value V1) from the grip part M12.

When the microphone M1 has not been set in the present disinfector 1, the microphone M1 is not in the determination region, so no reflected light from the microphone M1 is received by the light receiving element 151b.

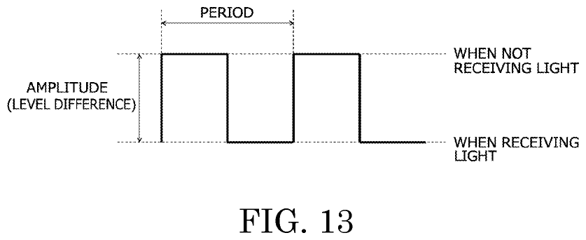

FIG. 13 is an enlarged schematic view illustrating an example of the pulsed electric signal from the light receiving element 151b.

In FIG. 13, a vertical axis indicates a voltage value and a horizontal axis indicates a time. FIG. 13 illustrates that a voltage value when infrared light (reflected light) is received (when receiving light) is lower than a voltage value when no infrared light (reflected light) is received (when not receiving light). The absolute value of the difference between the two voltage values is an amplitude value, that is, a signal level difference between a signal level when not receiving light and a signal level when receiving light. That is, the amplitude value is the absolute value of the difference between a voltage value when the light emitting element 151a emits light and a voltage value when the light emitting element 151a emits no light.

Referring now back to FIG. 11 and FIG. 12, next, the determination part 172 compares the amplitude value of the electric signal from the light receiving element 151b with the first threshold value V1 for each period of the electric signal, and determines whether the amplitude value is equal to or greater than the first threshold value V1 (S12).

The “first threshold value V1” is a threshold value set so that the amplitude value is always exceeded when the microphone M1 is in the determination region. The first threshold value V1 is set in advance based on, for example, the surface state (color, material, surface treatment, and the like) of a determination target (microphone M1), and is stored in the storage part 173. In this way, the first threshold value V1 is set based on the surface state of the microphone M1, so that the present disinfector 1 can determine the presence or absence of the microphone M1 with high accuracy in the first determination process S1.

When the amplitude value is equal to or greater than the first threshold value V1 (“Yes” at S12), the determination part 172 determines whether a state (hereinafter, referred to as a “first state”), in which the amplitude value is equal to or greater than the first threshold value V1, is continuous for a first time T1, starting from the period thereof (S13).

The “first time T1” is a time during which the first state is always continuous when the microphone M1 is in the determination region. The first time T1 is set in advance based on, for example, the light emission period of the infrared light, the degree of erroneous determination based on the installation environment of the present disinfector 1 (for example, brightness of sunlight and lighting, the presence or absence of infrared rays from an infrared remote controller, and the like), a time until the microphone M1 can be disinfected and sterilized after the microphone M1 is set in the present disinfector 1, and the like, and is stored in the storage part 173.

In the present embodiment, the determination part 172 measures the number (hereinafter, referred to as “the first number of consecutive times”) of periods in which the amplitude value is continuously equal to or greater than the first threshold value V1, and determines whether the first state is continuous for the first time T1 based on the first number of consecutive times. In such a case, the number of times serving as a threshold value for determination is a number (rounded up after the decimal point) obtained by dividing the first time T1 by the period (0.04 seconds) of the electric signal. That is, for example, when the first time T1 is set to 0.5 seconds, the determination part 172 determines that the first state is continuous for the first time T1 if the first number of consecutive times is 13 or more, and determines that the first state is not continuous for the first time T1 if the first number of consecutive times is less than 12.

When the first state is continuous for the first time T1 (“Yes” at S13), the determination part 172 determines that the microphone M1 is in the determination region (S14).

On the other hand, when the amplitude value is less than the first threshold value V1 (“No” at S12) or when the first state is not continuous for the first time T1 (“No” at S13), the determination part 172 determines that the microphone M1 is not in the determination region (S15).

In this way, in the first determination process S1, the light emitting element 151a emits infrared light with a frequency extremely lower than the carrier frequency band of the transmission infrared light. Therefore, even though the light receiving element 151b receives the transmission infrared light, the transmission infrared light is output from the light receiving element 151b like a noise component. As a consequence, the present disinfector 1 does not malfunction due to the transmission infrared light. Furthermore, the infrared light from the light emitting element 151a is pulsed light with a constant light emission period and a pulsed electric signal with a constant period is output from the light receiving element 151b having received reflected light. Therefore, the determination part 172 can indirectly acquire the period of the electric signal by measuring the first number of consecutive times. As a consequence, the present disinfector 1 can discriminate an electric signal based on light (light with an indefinite period or light that is not pulsed light) from the installation environment and an electric signal based on the reflected light of the infrared light from the light emitting element 151a. As a consequence, the malfunction of the present disinfector 1 based on the light from the installation environment is restrained.

Note that the determination part in the present invention may directly measure the duration of the first state, instead of the indirect determination of the elapsed time based on the first number of consecutive times.

Furthermore, the determination part in the present invention may acquire the period of the electric signal in the first state and determine whether the period matches the light emission period of the infrared light. In such a case, when the first state is continuous for the first time and the period matches the light emission period, the determination part in the present invention may determine that a microphone is in the determination region. According to such a configuration, since the present disinfector determines the presence or absence of a microphone only based on reflected light, the present disinfector does not determine that a microphone is present, based on light other than the reflected light (light from the installation environment).

In the first determination process S1, the determination execution time in an initial state is a first determination execution time (always). For example, in the process S12, when the amplitude value is not equal to or greater than the first threshold value V1 continuously for a predetermined time (for example, 10 minutes), the switch part 174 switches the determination execution time to a second determination execution time (periodically). As a consequence, the processing load of the determination part 172 is reduced. Furthermore, since an electric signal based on infrared light used as a carrier wave for wireless transmission is output in an extremely short time, the influence of the electric signal on the first determination process S1 is reduced. In such a case, for example, when the amplitude value is equal to or greater than the first threshold value V1, the switch part 174 switches the determination execution time to the first determination execution time.

### Second Determination Process

The “second determination process S2” is a process of determining the presence or absence of the microphone M in the determination region when the microphone M is in the determination region. That is, the second determination process S2 is a process of determining whether the microphone M to be disinfected and sterilized has been taken out from the present disinfector 1. The second determination process S2 is always executed after the determination that the microphone M is in the determination region in the first determination process S1.

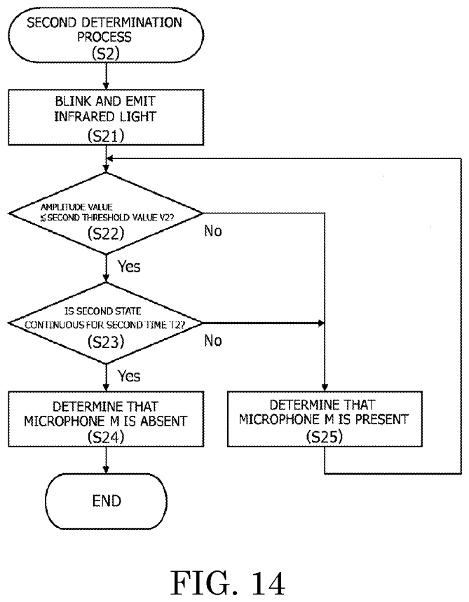

FIG. 14 is a flowchart illustrating the second determination process S2.

FIG. 15 is a schematic view illustrating an example of an electric signal from the light receiving element 151b in the second determination process S2.

First, the light emission control part 171 allows the light emitting element 151a to blink and emit infrared light that is pulsed light with a constant light emission period consecutively even after the first determination process S1 (521).

Next, when the determination part 172 determines that the microphone M1 is in the determination region, the determination part 172 compares the amplitude value with a second threshold value V2 for each period of an electric signal, and determines whether the amplitude value is equal to or less than the second threshold value V2 (S22).

The “second threshold value V2” is a threshold value set so that the amplitude value is always lower when the microphone M1 is not in the determination region. The second threshold value V2 is smaller than the first threshold value Vl. The second threshold value V2 is set in advance based on, for example, the surface state of the determination target (microphone M0 and the installation environment of the present disinfector 1, and is stored in the storage part 173. In this way, the second threshold value V2 is set based on the surface state of the microphone M1, so that the present disinfector 1 can determine the presence or absence of the microphone M1 with high accuracy in the second determination process S2. As described above, even when the microphone M1 is not in the determination region, the electric signal may have a minute amplitude value due to light reception of light from the installation environment, noise, and the like. Therefore, the second threshold value V2 is set to a value larger than “0”.

When the amplitude value is equal to or less than the second threshold value V2 (“Yes” at S22), the determination part 172 determines whether a state (hereinafter, referred to as a “second state”), in which the amplitude value is equal to or less than the second threshold value V2, is continuous for a second time T2, starting from the period thereof (S23).

The “second time T2” is a time during which the second state is always continuous when the microphone M1 is not in the determination region. The second time T2 is set in advance based on, for example, the blinking light emission period of the infrared light, the installation environment of the present disinfector 1, a time until disinfection and sterilization are not possible after the microphone M1 is taken out, and the like, and is stored in the storage part 173. In the present embodiment, the second time T2 is longer than the first time T1.

Note that the second time may be the same as the first time.

In the present embodiment, the determination part 172 measures the number (hereinafter, referred to as “the second number of consecutive times”) of periods in which the amplitude value is continuously equal to or less than the second threshold value V2, and determines whether the second state is continuous for the second time T2 based on the second number of consecutive times. In such a case, the number of times serving as a threshold value for determination is a number (rounded up after the decimal point) obtained by dividing the second time T2 by the period of the electric signal. That is, for example, when the second time T2 is set to 1 second, the determination part 172 determines that the second state is continuous for the second time T2 if the second number of consecutive times is 25 or more, and determines that the second state is not continuous for the second time T2 if the second number of consecutive times is less than 24.

When the second state is continuous for the second time T2 (“Yes” at S23), the determination part 172 determines that the microphone M1 is not in the determination region (S24).

On the other hand, when the amplitude value exceeds the second threshold value V2 (“No” at S22) or when the second state is not continuous for the second time T2 (“No” at S23), the determination part 172 determines that the microphone M1 is in the determination region (S25).

In this way, even in the second determination process S2, the light emitting element 151a emits infrared light with a frequency extremely lower than the carrier frequency band of the transmission infrared light. Therefore, the present disinfector 1 does not malfunction due to the transmission infrared light. Furthermore, the determination part 172 can indirectly acquire the period of the electric signal by measuring the second number of consecutive times. As a consequence, when the period of the electric signal is different from the blinking light emission period of the infrared light, the malfunction of the present disinfector 1 based on an electric signal with a period different from the blinking light emission period is restrained. Furthermore, the present disinfector 1 can discriminate an electric signal based on light (light with an indefinite period or light that is not pulsed light) from the installation environment and an electric signal based on the reflected light of the infrared light from the light emitting element 151a. Therefore, the malfunction of the present disinfector 1 based on the light from the installation environment is restrained.

### Output Adjustment Process

The “output adjustment process S3” is a process of adjusting (reducing) the light emission output of the light emitting element 151a when the microphone M is in the determination region.

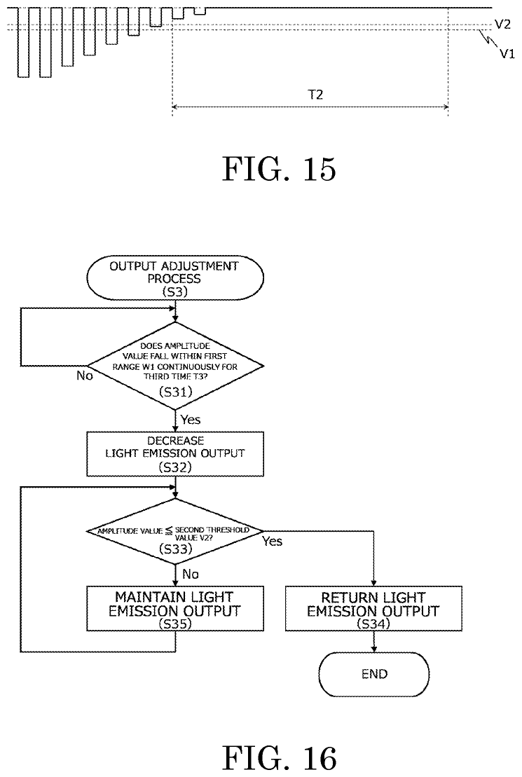

FIG. 16 is a flowchart illustrating the output adjustment process S3.

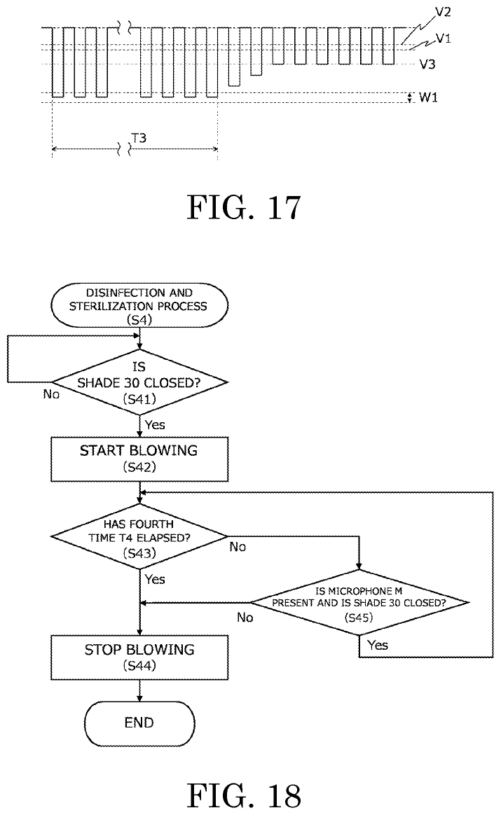

FIG. 17 is a schematic view illustrating an example of an electric signal from the light receiving element 151b in the output adjustment process S3.

First, when it is determined in the first determination process S1 that the microphone M1 is in the determination region, the determination part 172 determines whether the amplitude value falls within a first range W1 continuously for a third time T3 (S31).

The “third time T3” is a time during which a stable amplitude value is reliably continuous when the microphone M1 is in the determination region. The third time T3 is set in advance based on, for example, an amplitude value measured when the microphone M1 is in the determination region, and is stored in the storage part 173.

The “first range W1” is a range indicating a variation (fluctuation) in the amplitude value when the amplitude value is stable. The first range W1 is set in advance based on, for example, the amplitude value within a predetermined time measured when the microphone M1 is in the determination region, and is stored in the storage part 173.

When the amplitude value falls within the first range W1 continuously for the third time T3 (“Yes” at S31), the light emission control part 171 decreases the light emission output of the light emitting element 151a until the amplitude value reaches a third threshold value V3 (S32). On the other hand, when the amplitude value does not fall within the first range W1 continuously for the third time T3 (“No” at S31), the determination part 172 continuously executes the process S31.

The “third threshold value V3” is a threshold value indicating an amplitude value at which the first state can be reliably continuous even though the light emission output is made lower than the light emission output of the light emitting element 151a in the first determination process S1. The third threshold value V3 is set in advance based on, for example, the first threshold value V1 or the second threshold value V2, the variation in the amplitude value, and the like, and is stored in the storage part 173. That is, for example, when the variation in the amplitude value is ±5% of the first threshold value V1, the third threshold value V3 can be set to a value 1.2 to 1.5 times the first threshold value V1.

Note that the third threshold value may be set based on the second threshold value, instead of the first threshold value.

Next, the determination part 172 determines whether the amplitude value is equal to or less than the second threshold value V2 for each period (S33).

When the amplitude value is equal to or less than the second threshold value V2 (“Yes” at S33), the light emission control part 171 returns (increases the light emission output) the light emission output of the light emitting element 151a to the light emission output in the first determination process S1 (S34).

On the other hand, when the amplitude value is greater than the second threshold value V2 (“No” at S33), the light emission control part 171 maintains the light emission output of the light emitting element 151a at the third threshold value V3 (S35).

Note that the determination part in the present invention may determine whether it is determined that a microphone is not in the determination region in the second determination process, instead of the determination regarding whether the amplitude value is equal to or less than the second threshold value for each period in the process (S33). In such a case, the light emission control part in the present invention increases the light emission output when it is determined that the microphone is absent, and maintains the light emission output when it is determined that the microphone is present.

In this way, when the microphone M is not in the determination region, the present disinfector 1 emits infrared light with large light emission output in consideration of diffuse reflection off the surface of the microphone M, the installation environment, and the like. On the other hand, when the microphone M is in the determination region, the present disinfector 1 decreases the light emission output to the extent that the second determination process S2 can be executed. As a consequence, the current consumption of the light emitting element 151a in the present disinfector 1 is reduced.

### Disinfection and Sterilization Process

The “disinfection and sterilization process S4” is a process of disinfecting and sterilizing the microphone M when the microphone M is in the determination region. That is, the disinfection and sterilization process S4 is a process of disinfecting and sterilizing the microphone M set in the present disinfector 1.

FIG. 18 is a flowchart illustrating the disinfection and sterilization process S4.

First, the present disinfector 1 determines whether the shade 30 is closed (S41). The determination regarding the opening and closing of the shade 30 is performed by, for example, the body control part 18.

When the shade 30 is closed (“Yes” at S41), the body control part 18 controls the operations of the ozone generation part 12 and the fan 14 to start blowing the ozone wind (S42). As a consequence, the head parts M11 and M21 of the microphones M1 and M2 are disinfected and sterilized by the ozone wind that is blown from the blowing ports 11h1 to the space in the shade 30 and stays therein. On the other hand, when the shade 30 is opened (“No” at S41), the disinfection and sterilization process S4 returns to the process S41.

Next, the body control part 18 determines whether the elapsed time after the ozone generation part 12 and the fan 14 are operated has passed a fourth time T4 (S43). The “fourth time T4” is a time required for the ozone wind to disinfect and sterilize the head parts M11 and M21. The fourth time T4 is set in advance and stored in the body control part 18.

When the elapsed time has elapsed the fourth time T4 (“Yes” at S43), the body control part 18 controls the operations of the ozone generation part 12 and the fan 14 to stop blowing the ozone wind (S44). On the other hand, when the elapsed time has not elapsed the fourth time T4 (“No” at S43), the present disinfector 1 determines the presence or absence of the microphone M1 in the determination region and the opening and closing of the shade 30 (S45). The presence or absence of the microphone M1 is determined according to, for example, whether the first state is continuous and/or the second state is continuous.

When the microphone M1 is in the determination region and the shade 30 is closed (“Yes” at S45), the disinfection and sterilization process S4 returns to the process S43. On the other hand, when the microphone M1 is not in the determination region and the shade 30 is opened (“No” at S45), the disinfection and sterilization process S4 returns to the process S44.

### Summary

According to the embodiment described above, the present disinfector 1 includes the light emitting elements 151a and 161a, the light receiving elements 151b and 161b, and the determination part 172. Infrared light from the light emitting elements 151a and 161a is pulsed light with a predetermined blinking light emission period. The determination part 172 determines the presence or absence of the microphone M based on whether the first state, in which the amplitude value of an electric signal from the light receiving elements 151b and 161b is equal to or greater than the first threshold value V1, is continuous for the first time T1. In other words, the determination part 172 determines the presence or absence of the microphone M based on the amplitude value (signal level difference) of the electric signal and a duration during which the amplitude value is equal to or greater than the first threshold value V1. That is, the determination part 172 determines the presence or absence of the microphone M based on the duration of the first state as well as the magnitude of the amplitude value. As a consequence, it becomes difficult for the determination part 172 to determine that the microphone M is in the determination region based on light from the installation environment, which is sporadically and instantaneously received. Consequently, the malfunction of the present disinfector 1 due to the light from the installation environment is restrained. As a consequence, the present disinfector 1 can stably detect the presence or absence of the microphone M by the infrared light.

Furthermore, according to the embodiment described above, the determination part 172 determines whether the first state is continuous for the first time T1 based on the number (the first number of consecutive times) of periods in which the amplitude value is continuously equal to or greater than the first threshold value Vl. In other words, the determination part 172 also determines the presence or absence of the microphone M based on the period of the electric signal. According to such a configuration, when the period of the electric signal is different from the blinking light emission period of the infrared light, the malfunction of the present disinfector 1 based on an electric signal with a period different from the blinking light emission period is restrained. Furthermore, the present disinfector 1 can discriminate an electric signal based on the light (light with an indefinite period or light that is not pulsed light) from the installation environment and an electric signal based on the reflected light of the infrared light. Therefore, the malfunction of the present disinfector 1 based on the light from the installation environment is restrained. As a consequence, the present disinfector 1 can stably detect the presence or absence of the microphone M by the infrared light.

Moreover, according to the embodiment described above, when it is determined that the microphone M is present, the determination part 172 determines the presence or absence of the microphone M based on whether the second state, in which the amplitude value is equal to or less than the second threshold value V2, is continuous for the second time T2. According to such a configuration, it becomes difficult for the determination part 172 to determine that the microphone M is not in the determination region based on the light from the installation environment, which is sporadically and instantaneously received. Consequently, the malfunction of the present disinfector 1 due to the light from the installation environment is restrained. As a consequence, the present disinfector 1 can stably detect the presence or absence of the microphone M by the infrared light.

Moreover, according to the embodiment described above, the determination part 172 determines whether the second state is continuous for the second time T2 based on the number (the second number of consecutive times) of periods in which the amplitude value is continuously equal to or less than the second threshold value V2. According to such a configuration, when the period of the electric signal is different from the blinking light emission period of the infrared light, the malfunction of the present disinfector 1 based on an electric signal with a period different from the blinking light emission period is restrained. Furthermore, the present disinfector 1 can discriminate an electric signal based on the light from the installation environment and an electric signal based on the reflected light of the infrared light. Therefore, the malfunction of the present disinfector 1 based on the light from the installation environment is restrained. As a consequence, the present disinfector 1 can stably detect the presence or absence of the microphone M by the infrared light.

Moreover, according to the embodiment described above, the first time T1 is different from the second time T2. According to such a configuration, the determination accuracy (detection accuracy of the microphone M) of each of the first determination process S1 and the second determination process S2 and the reaction speed of the present disinfector 1 can be individually adjusted. That is, for example, in the present embodiment, the first time T1 is shorter than the second time T2. Therefore, the present disinfector 1 can detect the setting of the microphone M in the present disinfector 1 at a high speed with a certain degree of determination accuracy, and start disinfecting and sterilizing the microphone M. Furthermore, the present disinfector 1 can more reliably detect that the microphone M has been taken out from the present disinfector 1, and prevent the ozone wind from being unnecessarily blown.

Moreover, according to the embodiment described above, the first threshold value V1 is different from the second threshold value V2. When the first threshold value and the second threshold value are the same, if the amplitude value fluctuates in the vicinity of the first threshold value (second threshold value), the determination regarding the elapsed time of the second state can be started during the determination regarding the elapsed time of the first state in the first determination process S1 (determinations regarding the two elapsed times may conflict). However, according to such a configuration, even when the amplitude value is not stable and fluctuates relatively large, such confliction is avoided.

Moreover, according to the embodiment described above, the light emission control part 171 allows the light emission output when the determination part 172 determines that the microphone M is present to be lower than the light emission output when the determination part 172 determines that the microphone M is absent. According to such a configuration, when the microphone M is in the determination region, the present disinfector 1 decreases the light emission output to the extent that the second determination process S2 can be executed. As a consequence, the current consumption of the light emitting elements 151a and 161a in the present disinfector 1 is reduced.

Moreover, according to the embodiment described above, when the amplitude value falls within the first range W1 continuously for the third time T3, the light emission control part 171 decreases the light emission output. According to such a configuration, the present disinfector 1 can automatically reduce the current consumption of the light emitting elements 151a and 161a. Furthermore, the third threshold value V3 can be set to a value close to the first threshold value V1 or the second threshold value V2, resulting in a reduction in power consumption.

Moreover, according to the embodiment described above, when the amplitude value is not equal to or greater than the first threshold value V1 continuously for a predetermined time, the switch part 174 switches between the first determination execution time and the second determination execution time. According to such a configuration, the processing load of the determination part 172 is reduced. Furthermore, the influence of the infrared light from the installation environment in the first determination process S1 is reduced. As a consequence, the present disinfector 1 can stably detect the presence or absence of the microphone M by the infrared light.

Moreover, according to the embodiment described above, the sensor covers 152 and 162 include the long groove 152c arranged in the first surface 152a so as to separate the light emitting elements 151a and 161a and the light receiving elements 151b and 161b in the front view. According to such a configuration, an air layer (long groove 152c) having a refraction index different from those of the sensor covers 152 and 162 is formed inside the sensor covers 152 and 162. As a consequence, infrared light reflected in the sensor covers 152 and 162 and directed toward the light receiving elements 151b and 161b is reflected again in the sensor covers 152 and 162 by the air layer. That is, the infrared light reflected in the sensor covers 152 and 162 is not received by the light receiving elements 151b and 161b. Consequently, the erroneous detection of the light receiving elements 151b and 161b is restrained, and the malfunction of the present disinfector 1 is restrained. As a consequence, the present disinfector 1 can stably detect the presence or absence of the microphone M by the infrared light.

Moreover, according to the embodiment described above, the frequency band of the infrared light is 10 Hz to 100 Hz. According to such a configuration, the light emitting elements 151a and 161a emit infrared light with a frequency extremely lower than the carrier frequency band of the transmission infrared light. Therefore, even though the light receiving elements 151b and 161b receive the transmission infrared light, the transmission infrared light is output from the light receiving elements 151b and 161b like a noise component. As a consequence, the malfunction of the present disinfector 1 due to the transmission infrared light does not occur. As a consequence, the present disinfector 1 can stably detect the presence or absence of a microphone M by the infrared light. Moreover, the malfunction of an external device (for example, a television and an air conditioner operated by an infrared remote controller) due to the infrared light from the light emitting elements 151a and 161a also does not occur.

Note that the disinfection part in the present invention may be configured by a light source that emits ultraviolet rays, or may be configured to spray steam or alcohol. In such a case, the filter and the fan may not be provided in the present disinfector as needed.

Furthermore, the present disinfector may be individually provided with a support part for supporting a microphone.

Moreover, the number of microphones to be disinfected and sterilized by the present disinfector is not limited to the present embodiment (two).

Moreover, the present disinfector may not be provided with the switch part. In such a case, the determination execution time of the present disinfector is maintained at the first determination execution time.

Moreover, the present disinfector may not execute the output adjustment process.

Moreover, a determination target of the first sensor and the second sensor in the present invention is not limited to a microphone.

Moreover, the first determination process and the second determination process can also be used to determine the presence or absence of an object other than a microphone.

1. A microphone disinfector comprising:
a disinfection part that disinfects and sterilizes a microphone;
a light emitting part that emits infrared light to a region where the microphone is arranged;
a light receiving part that receives reflected light of the infrared light and generates an electric signal based on the received light;
a determination part that determines presence or absence of the microphone in the region based on the electric signal; and
a disinfection control part that controls an operation of the disinfection part based on a determination result of the determination part, wherein
the infrared light is pulsed light with a predetermined blinking light emission period, and
the determination part determines whether an amplitude value for each period of the electric signal is equal to or greater than a first threshold value, and determines the presence or absence of the microphone based on whether a first state, in which the amplitude value is equal to or greater than the first threshold value, is continuous for a first time.

2. The microphone disinfector according to claim 1, wherein the determination part determines that the microphone is present when the first state is continuous for the first time, and determines that the microphone is absent when the first state is not continuous for the first time.

3. The microphone disinfector according to claim 2, wherein the determination part determines whether the first state is continuous for the first time based on the number of periods in which the amplitude value is continuously equal to or greater than the first threshold value.

4. The microphone disinfector according to claim 2, wherein the determination part
determines whether the period of the electric signal matches the blinking emission period of the infrared light,
determines that the microphone is present when the first state is continuous for the first time and the period matches the blinking light emission period, and
determines that the microphone is absent when the period does not match the blinking light emission period.

5. The microphone disinfector according to claim 2, wherein, when the determination part determines that the microphone is present, the determination part
determines whether the amplitude value is equal to or less than a second threshold value, and
determines the presence of absence of the microphone based on whether a second state, in which the amplitude value is equal to or less than the second threshold value, is continuous for a second time.

6. The microphone disinfector according to claim 5, wherein the determination part
determines that the microphone is present when the second state is continuous for the second time, and
determines that the microphone is absent when the second state is not continuous for the second time.

7. The microphone disinfector according to claim 6, wherein the determination part determines whether the second state is continuous for the second time based on the number of periods in which the amplitude value is continuously equal to or less than the second threshold value.

8. The microphone disinfector according to claim 5, wherein the first time is different from a second time, and/or the first threshold value is different from the second threshold value.

9. The microphone disinfector according to claim 5, further comprising a light emission control part that controls light emission output of the infrared light emitted from the light emitting element based on the determination result, wherein
the light emission control part decreases the light emission output when the determination part determines that the microphone is present to be lower than the light emission output when the determination part determines that the microphone is absent based on the first threshold value or the second threshold value.

10. The microphone disinfector according to claim 9, wherein
when the determination part determines that the microphone is present, the determination part determines whether the amplitude value falls within a predetermined range continuously for a third time, and
when the amplitude value falls within the predetermined range continuously for the third time, the light emission control part decreases the light emission output to be lower than the light emission output when the determination part determines that the microphone is absent.

11. The microphone disinfector according to claim 1, further comprising a switch part that switches a determination execution time that is a time during which the determination part continuously executes a determination regarding whether the amplitude value is equal to or greater than the first threshold value, wherein
the switch part switches the determination execution time based on a time during which the amplitude value is continuously not equal to or greater than the first threshold value.

12. The microphone disinfector according to claim 11, wherein the determination execution time includes a first determination execution time at which the determination part always executes the determination and a second determination execution time at which the determination part periodically executes the determination.

13. The microphone disinfector according to claim 1, further comprising a cover that covers a front surface of each of the light emitting part and the light receiving part and allows the infrared light and the light to pass therethrough, wherein
the light emitting part and the light receiving part are integrally configured, and
the cover includes:
a first surface that faces the front surface of each of the light emitting part and the light receiving part;
a second surface parallel to the first surface; and
a long groove arranged in the first surface so as to separate the light emitting part and the light receiving part in a front view of the light emitting part and the light receiving part.

14. The microphone disinfector according to claim 13, wherein a depth of the long groove is 2/5 or larger of a thickness between the first surface and the second surface of the cover.

15. The microphone disinfector according to claim 13, wherein the long groove is recessed in a rectangular shape from the first surface toward the second surface in a cross-sectional view parallel to a short direction of the long groove.

16. The microphone disinfector according to claim 1, wherein a frequency band of the infrared light is 10 Hz to 100 Hz.
